(19) United States
(12) Patent Application Publication
(10) Pub. No.: US 2025/0090003 A1
(43) Pub. Date: Mar. 20, 2025

(54) MULTI-LUMEN TUBING SYSTEM FOR USE WITH AN ENDOSCOPE

(71) Applicant: Motus GI Medical Technologies Ltd., Tirat HaCarmel (IL)

(72) Inventors: Iddo SALMAN, Tirat Carmel (IL); Rephael SOMMER, Nesher (IL); Mark POMERANZ, Leesburg, VA (US)

(73) Assignee: Motus GI Medical Technologies Ltd., Tirat HaCarmel (IL)

(21) Appl. No.: 18/727,126

(22) PCT Filed: Feb. 5, 2023
(86) PCT No.: PCT/IL2023/050126

§ 371 Date: Jul. 8, 2024

Related U.S. Application Data
(60) Provisional application No. 63/306,538, filed on Feb. 4, 2022.

(51) Int. Cl. A61B 1/012; A61B 1/00; A61B 1/31

(52) U.S. Cl. CPC A61B 1/012; A61B 1/00078; A61B 1/00121; A61B 1/31

(57) ABSTRACT

A multi-lumen insertion tube for use with an endoscope, the multi-lumen insertion tube having a circumferential outer wall defining a main inner lumen, and a plurality of inner septa dividing the main inner lumen into a plurality of lumens. The circumferential outer wall is configured to expand radially outwardly when positive pressure is applied through the insertion tube, and to at least partially collapse against at least one of the inner septa when negative pressure is applied through the insertion tube, such that further collapse of the circumferential wall is prevented by at least one of the inner septa.

### RELATED APPLICATION/S

This application claims the benefit of priority of U.S. Provisional Patent Application No. 63/306,538 filed on 4 Feb. 2022, the contents of which are incorporated herein by reference in their entirety.

### FIELD AND BACKGROUND OF THE INVENTION

The present invention, in some embodiments thereof, relates to a tubing system for use with an endoscope and, more particularly, but not exclusively, to a tubing system in which tubes are divided by inner septa into multiple lumens.

### SUMMARY OF THE INVENTION

According to an aspect of some embodiments there is provided a multi-lumen insertion tube for use with an endoscope, comprising:
a circumferential outer wall defining a main inner lumen;
a plurality of inner septa dividing the main inner lumen into a plurality of lumens;
wherein the circumferential outer wall is configured to expand radially outwardly when positive pressure is applied through the insertion tube, and to at least partially collapse against at least one of the inner septa when negative pressure is applied through the insertion tube, such that further collapse of the circumferential wall is prevented by the at least one of the inner septa.

In some embodiments, the at least one of the inner septa defines a triangular wall, and the circumferential outer wall leans against an apex of the triangular wall when negative pressure is applied through the insertion tube.

In some embodiments, the triangular wall is formed of wall segments short enough to be substantially undeformable under the negative pressure.

In some embodiments, the circumferential wall is configured to expand radially outwardly when positive pressure applied through the insertion tube.

In some embodiments, the plurality of inner lumens include at least a first inner lumen and a second inner lumen, wherein flow in each of the first and second lumens is independently controlled.

In some embodiments, the first inner lumen serves for irrigation and the second inner lumen serves for suction.

In some embodiments, a cross section area of the multi-lumen insertion tube is sized for insertion into a colon.

In some embodiments, a thickness of the circumferential wall is between 0.5 mm and 1 mm and a thickness of the inner septa is between 0.3 mm and 0.8 mm.

In some embodiments, the circumferential outer wall comprises a corrugation along at least a segment of the length of the multi-lumen insertion tube.

In some embodiments, there is provided a tubing system for use with an endoscope, comprising:
a multi-lumen insertion tube, the multi-lumen insertion tube shaped and sized for insertion into a body lumen;
a multi-lumen umbilical tube configured for extending externally to the body, the multi-lumen umbilical tube comprising a plurality of inner lumens; and
a coupling interface which couples a proximal end of the insertion tube to a distal end of the umbilical tube, the coupling interface shaped to fluidly connect the plurality of inner lumens of the insertion tube to the plurality of inner lumens of the umbilical tube.

In some embodiments, a proximal end of the multi-lumen insertion tube is configured to connect to a pressure source which supplies positive and/or negative pressure.

In some embodiments, the multi-lumen umbilical tube is larger in cross-sectional area than the multi-lumen insertion tube.

In some embodiments, the coupling interface is formed as a connector comprising a first portion in which the proximal end of the insertion tube is received, and a second portion in which the distal end of the umbilical tube is received.

In some embodiments, the tubing system further comprises a loop shaped attachment extending laterally outwardly from the coupling interface for insertion of an endoscope through.

According to an aspect of some embodiments there is provided a method of setting up a tubing system for use with an endoscope, the tubing system comprising a multi-lumen insertion tube configured to connect at its proximal end to a multi-lumen umbilical tube, the method comprising:
aligning and coupling the multi-lumen insertion tube to an insertion tube of the endoscope;
connecting, via a connector, the multi-lumen insertion tube to the multi-lumen umbilical tube;
attaching the connector to the endoscope; and
connecting the multi-lumen umbilical tube to a working station of the endoscope.

In some embodiments, aligning and coupling the multi-lumen insertion tube to the insertion tube of the endoscope comprises one or both of holding, via a holder, the distal ends of the multi-lumen insertion tube and the endoscope insertion tube together; and covering the multi-lumen insertion tube and the endoscope insertion tube along their length by a sleeve which holds them together.

In some embodiments, attaching the connector to an endoscope comprises placing a loop shaped attachment extending from the connector around the endoscope.

According to an aspect of some embodiments there is provided a method of operating a tubing system coupled to a colonoscope, the tubing system including a multi-lumen insertion tube, the method comprising:
introducing an insertion tube of the colonoscope along with the multi-lumen insertion tube of the tubing system into the colon, side-by-side;
applying one of suction and purging via the multi-lumen insertion tube; wherein during suction, inner septa of the multi-lumen insertion tube prevent the multi-lumen insertion tube from collapsing, and wherein during purging an outer wall of the multi-lumen insertion tube expands radially outwardly.

In some embodiments, the method further comprises removing the colonoscope insertion tube and the multi-lumen insertion tube from the colon, detaching the tubing system from the colonoscope insertion tube and disposing the tubing system.

According to an aspect of some embodiments there is provided a multi-lumen insertion tube for use with an endoscope, comprising:
a circumferential outer wall defining a main inner lumen;
a plurality of inner septa dividing the main inner lumen into a plurality of lumens;
wherein along a segment of a length of the insertion tube the circumferential outer wall is corrugated, having ridges and grooves.

In some embodiments, the ridges and grooves are shaped and sized to provide for local expansion at multiple axial locations along the segment.

In some embodiments, the segment is more flexible than one or more other longitudinal segments of the insertion tube.

In some embodiments, when the multi-lumen insertion tube is inserted alongside an endoscope insertion tube, the section is axially aligned with a bendable section of the endoscope insertion tube.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

Implementation of the method and/or system of embodiments of the invention can involve performing or completing selected tasks manually, automatically, or a combination thereof. Moreover, according to actual instrumentation and equipment of embodiments of the method and/or system of the invention, several selected tasks could be implemented by hardware, by software or by firmware or by a combination thereof using an operating system.

For example, hardware for performing selected tasks according to embodiments of the invention could be implemented as a chip or a circuit. As software, selected tasks according to embodiments of the invention could be implemented as a plurality of software instructions being executed by a computer using any suitable operating system. In an exemplary embodiment of the invention, one or more tasks according to exemplary embodiments of method and/or system as described herein are performed by a data processor, such as a computing platform for executing a plurality of instructions. Optionally, the data processor includes a volatile memory for storing instructions and/or data and/or a non-volatile storage, for example, a magnetic hard-disk and/or removable media, for storing instructions and/or data. Optionally, a network connection is provided as well. A display and/or a user input device such as a keyboard or mouse are optionally provided as well.

### BRIEF DESCRIPTION OF THE SEVERAL VIEWS OF THE DRAWING(S)

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIG. 1 is a block diagram of a tubing system for use with an endoscope, according to some embodiments;

FIG. 2 is a flowchart of a method of setting up and operating a tubing system for use with an endoscope (e.g. a colonoscope), according to some embodiments;

FIGS. 3A-C show the tubing system (FIG. 3A), the tubing system mounted onto a colonoscope (FIG. 3B), the tubing system and colonoscope connected to a tubing system working station and a colonoscope working station respectively (FIG. 3C), according to some embodiments;

FIG. 4 is a cross section view of a multi-lumen insertion tube, according to some embodiments;

FIGS. 5A-C are cross section views of the multi-lumen insertion tube of FIG. 4 during static mode, suction mode, and purging mode, according to some embodiments;

FIGS. 6A-C show various cross sections of a multi-lumen umbilical tube, according to some embodiments;

FIG. 7 shows a corrugated section of the multi-lumen insertion tube, according to some embodiments;

FIGS. 8A-C schematically show a connector for coupling a multi-lumen insertion tube to a multi-lumen umbilical tube and for mounting the assembled tubing system onto an endoscope, according to some embodiments;

FIGS. 9A-D are detailed views of a connector for coupling a multi-lumen insertion tube to a multi-lumen umbilical tube and for mounting the assembled tubing system onto an endoscope, shown in an open state (FIGS. 9A-B) and a closed state (FIGS. 9C-D), according to some embodiments;

FIGS. 10A-E are alternative cross section profiles of a multi lumen insertion tube, according to some embodiments; and

FIGS. 11A-C are perspective views of an endoscope having adhesive strips for connecting a multi-lumen insertion tube or a multi-lumen umbilical tube, according to some embodiments.

### DESCRIPTION OF SPECIFIC EMBODIMENTS OF THE INVENTION

The present invention, in some embodiments thereof, relates to a tubing system for use with an endoscope and, more particularly, but not exclusively, to a tubing system in which tubes are divided by inner septa into multiple lumens. Some embodiments relates to a tubing system used with a colonoscope and configured for insertion, at least in part, into a colon, alongside a colonoscope. A potential advantage of a multi-lumen tube as compared, for example, to use of a plurality of separate smaller tubes may include an effectively smaller cross sectional area, where a single inner wall actually serves as a wall of two lumens as opposed to wall of a single lumen in the construction of separate tubing. The reduced cross sectional area may facilitate maneuvering of the tube inside the body lumen, for example through curves. Another potential advantage of a multi-lumen tube (such as compared to an arrangement of multiple smaller separate tubes) may include reduced complexity in assembling and introducing of the tube(s) into the body lumen.

A broad aspect of some embodiments relates to reducing or preventing collapse of a tube inserted into a body lumen, while maintaining flexibility of the tube to enable navigation, steering and bending inside narrow body lumens, such as the colon. In some embodiments, collapse reduction/prevention is via one or more inner walls of the tube which are positioned to support the outer walls of the tube, optionally at one or more locations which are prone to deformation, for example when suction (vacuum) is applied through the tube. In some embodiments, due to the support provided by the inner walls of the tube, an outer wall of the tube can be relatively thin, potentially contributing to the tube's flexibility and pliancy. In some embodiments, each of the inner walls forms a small enough segment which is less (or not) prone to deformation under pressure, for example, the applying of vacuum, for example as compared to longer sections of the perimeter (outer wall) of the tube which, if not supported, may deform or collapse inwardly under pressure.

An aspect of some embodiments relates to a multi-lumen insertion tube which can be inserted alongside an endoscope into a body lumen, the multi-lumen insertion tube comprising inner septa which divide a lumen of the tube into a plurality (e.g. 2, 3, 4, 5 or a higher number) of inner lumens. In some embodiments, the inner lumens form elongate channels extending along the tube long axis.

In an exemplary structure, inner walls inside the tube form a triangular configuration, where an apex of the triangle serves as a leaning point for an outer wall of the tube if and when the outer wall deforms inwardly, for example when suction is applied through the tube. By supporting the outer wall, further collapse of the outer wall is prevented, maintaining an open volume in the tube in which fluid and/or matter may continue to pass. In some embodiments, support provided by the inner walls of the tube to the outer wall of the tube is shaped and positioned to maintain at least 60%, at least 70%, at least 90% or intermediate, larger or smaller percentage of the cross sectional area of the tube (such as compared to a static, non-collapsed or deformed state of the tube) open.

In some embodiments, flow through the inner channels of the insertion tube is independently controlled. In some embodiments, inner channels of the multi-lumen insertion tube serve different functions, for example, one channel is used for irrigation, while another channel is used for suction (e.g. for evacuation of fluid and/or fecal matter from the colon). In some embodiments, an inner channel is used for introducing of tools through, potentially serving as an additional working channel to the colonoscope working channel. Examples of tools for insertion through the inner channel may include a snare, an anchor, a grasper, a marking probe, a jaw-tool and/or others.

An aspect of some embodiments relates to a tubing system for use with an endoscope, the tubing system including a multi-lumen insertion tube placed in fluid communication with a multi-lumen umbilical tube. In some embodiments, the insertion tube of the tubing system is configured to extend alongside a colonoscope insertion tube, and the umbilical tube of the tubing system is configured to extend alongside an umbilical tube of the colonoscope. In some embodiments, each of the inner lumens of the insertion tube is respectively connected to a corresponding inner lumen of the umbilical tube.

In some embodiments, the multi-lumen umbilical tube is connected, at its distal end, to a proximal end of the multi-lumen insertion tube, and at its distal end to a working station (optionally, the colonoscope working station). In some embodiments, a pressure source (e.g. a pump, optionally configured at the working station) supplies proximally directed pressure and/or distally directed pressure into the tubing system. Optionally, different pressures (such as in magnitude and/or direction) are supplied through different inner lumens of the tubing system.

In some embodiments, the multi-lumen insertion tube is connected to the multi-lumen umbilical tube via a connector which forms fluid communication between the respective inner lumens. Alternatively, the insertion tube and the umbilical tube are integrally formed as a single conduit. A potential advantage of connecting the multi-lumen insertion tube to the multi-lumen umbilical tube via a connector may include facilitating the attachment (and/or detachment) of the tubing system as a whole onto the colonoscope.

In some embodiments, the insertion tube and umbilical tube are different in profile, for example, differ from each other in one or more of: a total cross sectional area, a rigidity of the tube, a thickness of the outer and/or inner walls of the tube, and others. In an example, the umbilical tube, as it extends externally to the patient body, is more rigid and larger in cross sectional area than the insertion tube. In some embodiments, the connector configured at the attachment between the tubes acts as an interface between the two different tube profiles. A potential advantage of a removable connection between the insertion tube and the umbilical tube may include that the insertion tube can be locally replaced without requiring the replacement of the whole tubing line.

An aspect of some embodiments relates to an insertion tube comprising a corrugated outer wall along at least a portion of the length of the tube. In some embodiments, the corrugated outer wall includes ridges and grooves which provide for local expansion (e.g. in a radially outward direction and/or an axial direction) at multiple axial locations along the corrugated section. A potential advantage of a corrugated section may include improved bending properties of the tube, optionally allowing for retroflection. Another potential advantage of a corrugated section may include that the local radial expansion may facilitate clearing of blockage, for example by purging applied through the tube. For example, since the corrugated section may allow for local radial expansion at each of multiple axial locations, it may be easier to push or pass a blockage through, optionally without blocking the full cross section area of the corrugated section. Another potential advantage of a corrugated section is that flexibility of the section may be improved (such as relative to non-corrugated sections), yet without comprising the integrity of the multiple inner lumens inside the insertion tube. For example, although the outer corrugated surface may provide additional flexibility or bending ability, the inner lumens remain substantially unaffected in cross-sectional area, such as due to the inner septa.

In some embodiments, when the tubing system insertion tube is introduced alongside the endoscope insertion tube, the corrugated section is axially aligned with at least a portion of a bendable section of the endoscope insertion tube.

Before explaining at least one embodiment of the invention in detail, it is to be understood that the invention is not necessarily limited in its application to the details of construction and the arrangement of the components and/or methods set forth in the following description and/or illustrated in the drawings and/or the Examples. The invention is capable of other embodiments or of being practiced or carried out in various ways.

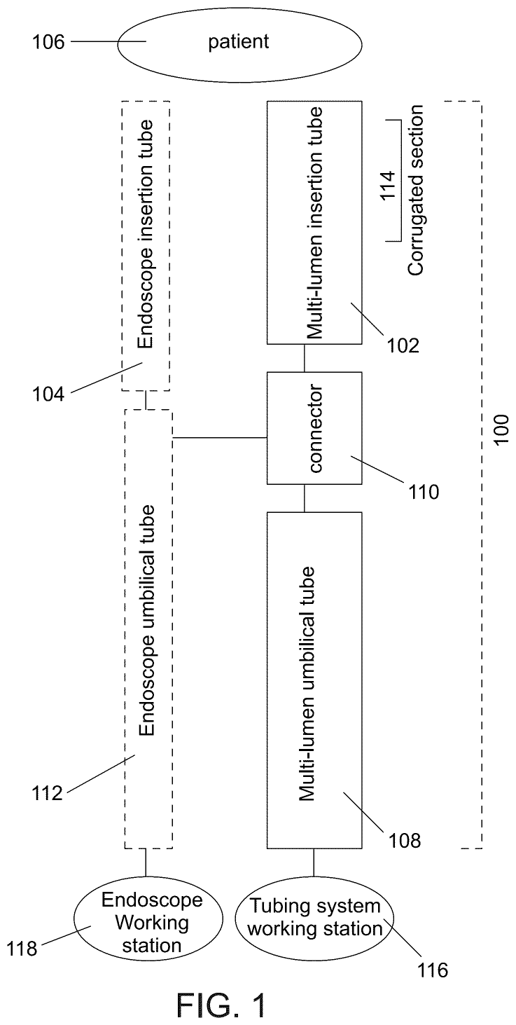

Referring now to the drawings, FIG. 1 a block diagram of a tubing system for use with an endoscope, according to some embodiments.

In some embodiments, at least a portion of the tubing system is introduced with the endoscope into a body lumen, for example, into the GI tract (e.g. into the colon), into the esophagus, into portions of the stomach and/or other.

In some embodiments, the tubing system is configured for suction and/or irrigation and/or purging action. In some embodiments, the tubing system provides for removal of matter from the body, for example, for cleaning fecal matter from a colon.

In some embodiments, tubing system 100 comprises a multi-lumen insertion tube 102, which is insertable along with a portion of an endoscope (an endoscope insertion tube 104) into a patient body 106. In some embodiments, insertion tube 102 is relatively small in diameter and is flexible enough to enable maneuvering of the tube along with the endoscope inside the body.

In some embodiments, insertion tube 102 is comprised of an extruded tube which is internally divided into a plurality of inner lumens, for example by inner walls or septa. In some embodiments, the inner lumens extend axially along the length of the insertion tube. In an example, insertion tube 102 comprises at least 2 inner lumens, 3 inner lumens, 4 inner lumens, 5 inner lumens or intermediate, larger or smaller number. In some embodiments, different inner lumens serve different functions, for example, one lumen is used for irrigation (i.e. for introducing fluid into the colon) and another lumen is used for suction (such as suction of matter and/or body fluids from the colon and externally to the body) and/or purging (such as for pushing away a blockage formed in the tube).

In some embodiments, a section 114 of the insertion tube 102 is corrugated on the external surface of the tube. In some embodiments, the corrugated section improves flexibility of the tube and facilitates bending and steering.

In some embodiments, tubing system 100 comprises a multi-lumen umbilical tube 108. In some embodiments, umbilical tube 108 is comprised of an extruded tube which is internally divided into a plurality of inner lumens, for example by inner walls or septa. In an example, umbilical tube 108 comprises at least 2 inner lumens, 3 inner lumens, 4 inner lumens, 5 inner lumens or intermediate, larger or smaller number. In some embodiments, different inner lumens serve different functions, for example, one lumen is used for irrigation (i.e. for introducing fluid into the colon) and another lumen is used for suction (such as suction of matter and/or body fluids from the colon) and/or purging (such as for pushing away a blockage formed in the tube). In some embodiments, the number of inner lumens in insertion tube 102 is similar to the number of inner lumens in umbilical tube 108. Alternatively, in some embodiments, the number of inner lumens in insertion tube 102 is different than the number of inner lumens in the umbilical tube 108, for example an additional lumen that starts at a connector 110 and extends either to the distal end (i.e. through the insertion tube) or to the proximal end (i.e. through the umbilical tube).

In some embodiments, a cross-sectional area of the umbilical tube is larger than that of insertion tube 102 (which is made possible since the umbilical tube is not introduced into the body). In some embodiments, an external wall of the umbilical tube 108 is thicker than that of the insertion tube 102. In some embodiments, the umbilical tube 108 is connected at its proximal end to a tubing system working station 116.

In some embodiments, umbilical tube 108 is coupled by the connector 110 to the insertion tube 102, proximally to the insertion tube. In some embodiments, connector 110 includes an inner interface for connecting the inner lumens of insertion tube 102 with the respective inner lumens of umbilical tube 108 such that uninterrupted fluid communication is formed between the lumens.

In some embodiments, connector 110 is configured to couple the connected insertion tube 102 and umbilical tube 108 to the endoscope, for example to an endoscope umbilical tube 112 and/or to the endoscope insertion tube 104. In some embodiments, attachment to the endoscope by connector 110 is external to the endoscope and external to tubes 102 and 108. In some embodiments, the connector comprises a clip, a tie (e.g. a Velcro tic), a wrapping tape and/or other means for externally attaching the tubing system to the endoscope. Optionally, connector 110 is shaped to provide for axial sliding of the endoscope relative to the tubing system or vice versa.

In some embodiments, one or more sensors are incorporated at the connector, for example for sensing pressure, flow and/or other parameters within inner lumens of the tubing system and/or within inner lumens of the endoscope system.

It is noted that in some embodiments, two separate connectors may be used: one for coupling the insertion tube 102 to the umbilical tube 108, and another for coupling the tubing system to the endoscope.

In some embodiments, insertion tube 102 and endoscope insertion tube 104 are coupled to each other at their distal ends and optionally along their length, for example by an external sleeve (e.g. a nylon sheath).

In some embodiments, the sleeve is non-uniform, for example comprising openings or portions that do not include material. For example, the sleeve comprises a mesh, multiple elongate stripes, and/or other non-uniform profiles. In an example, the sleeve is formed as a mesh including apertures having, for example, rounded or rectangular cross sectional profiles. A potential advantage of a mesh sleeve may include obtaining an improved grip of the endoscope(s) and add-on tube, and may potentially reduce interference of the sleeve with bending of the endoscope. Another potential advantage of a mesh sleeve may include a reduced contact surface with the inner walls of the GI tract, for example as compared to a sleeve that is uniform, for example, that has no radially outward facing apertures.

In some embodiments, the endoscope tubing (endoscope insertion tube 104, endoscope umbilical tube 112) is connected at its proximal end to an endoscope working station 118.

It is noted that in some embodiments a single working station which is configured for dually activating the tubing system and the endoscope may be used.

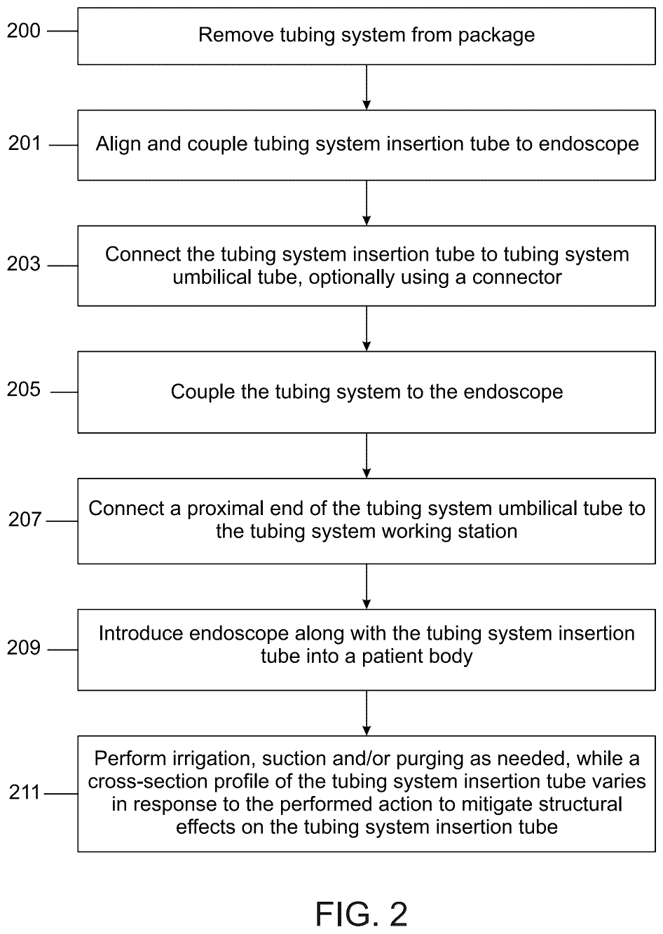

FIG. 2 is a flowchart of a method of setting up and operating a tubing system for use with an endoscope, according to some embodiments.

In some embodiments, set up of the tubing system is performed prior to the endoscopic procedure, for example by a physician, surgeon, nurse, technician or other certified personnel.

In some embodiments, the tubing system is removed from its packaging (200). Optionally, the tubing system is intended for single use (disposed following the procedure).

In some embodiments, the insertion tube of the tubing system is aligned and coupled to the endoscope (201), for example to the endoscope insertion tube. In some embodiments, a distal head of the tubing system insertion tube is coupled to a distal head of the endoscope insertion tube. Optionally, a head configured at a distal end of the tubing system insertion tube head is shaped to define a recess (e.g. a basket shaped recess) in which the distal head of the endoscope insertion tube is received.

In some embodiments, the tubing system insertion tube and the endoscope insertion tube are aligned to extend parallel and adjacent each other, at least along a portion of their lengths. In some embodiments, a cover sleeve is pulled over the aligned insertion tubes, holding the two insertion tubes together and protecting them. In some embodiments, the cover sleeve is attached, at its proximal end, to a ring clement which when pulled aligns the insertion tubes relative to each other, and dresses the cover sleeve over the insertion tubes together.

In some embodiments, the tubing system insertion tube is connected to the tubing system umbilical tube (203), for example via a connector mounted or received on a proximal end of the insertion tube. In some embodiments, the connector connects the inner lumens of the insertion tube and the umbilical tube such that fluid communication is provided between them.

In some embodiments, optionally using the same connector and/or a different connector, the tubing system (insertion tube and umbilical tube) is coupled to the endoscope (205). Connecting of the tubing system to the endoscope may be performed at one or more axial locations along the endoscope, for example along the endoscope umbilical tube, the endoscope handle, along a proximal portion of the endoscope insertion tube, and/or other locations. In some embodiments, connecting is such that tubing system tubes are held adjacent and in alignment with the endoscope tubes.

In some embodiments, a proximal end of the tubing system umbilical tube is connected to a tubing system working station (207). In some embodiments, one or more pressure sources (e.g. pumps) in the working station are configured to set and/or modify the pressure inside the tubing system, for example providing for changing the direction of the applied pressure (e.g. suction vs. purging), and/or for modifying (increasing or decreasing) a pressure gradient inside the tubing system.

In some embodiments, the endoscope (the endoscope insertion tube) along with the tubing system insertion tube are at least partially introduced into the patient body (209), for example, into the GI tract.

During the endoscopic procedure, one or more of the following actions may be performed via the tubing system: irrigation (introducing of fluids (e.g. saline, water) into the body, for example for clearing out the field of view; suction (such as of fecal matter, fluids, and/or other) which removes matter away from the body; purging (such as when blockage formed within the tubing system, and/or when blockage (e.g. by tissue) is formed at entry to the insertion tube, with the purging applying distally directed pressure which pushes the blockage away). In some embodiments, suction, irrigation and/or purging actions are performed while a cross-section profile of the multi-lumen insertion tube of the tubing system varies in response to the performed action to mitigate structural effects on the insertion tube (211). In some embodiments, the variation in tube cross section includes a change (e.g. deformation) of an outer wall portion of the insertion tube and/or of an inner septum of the insertion tube. In some embodiments, in response to the applied action, the outer wall portion and/or the inner septum (or a portion of it) move between at least a first position and a second position, resulting in a change of the insertion tube cross section layout. In some embodiments, the variation in cross-section is temporary and once a certain action is ceased, the outer and inner walls of the insertion tube elastically bounce back to their initial layout.

In some embodiments, flow within an inner lumen of the tubing system is reversible and/or otherwise variably controllable. Optionally, evacuation flow is controlled to reduce or prevent blockages during use with reduced need to interrupt a procedure.

In some embodiments, one or more sensors detect parameters of the flow within the tubing system. Optionally, one or more sensors are mounted inside an inner lumen for detecting. for example, flow, flow rate, pressure, a volume of matter, and/or other. In some embodiments, one or more sensors are mounted outside the tubing system, for example for detecting pressure in the colon.

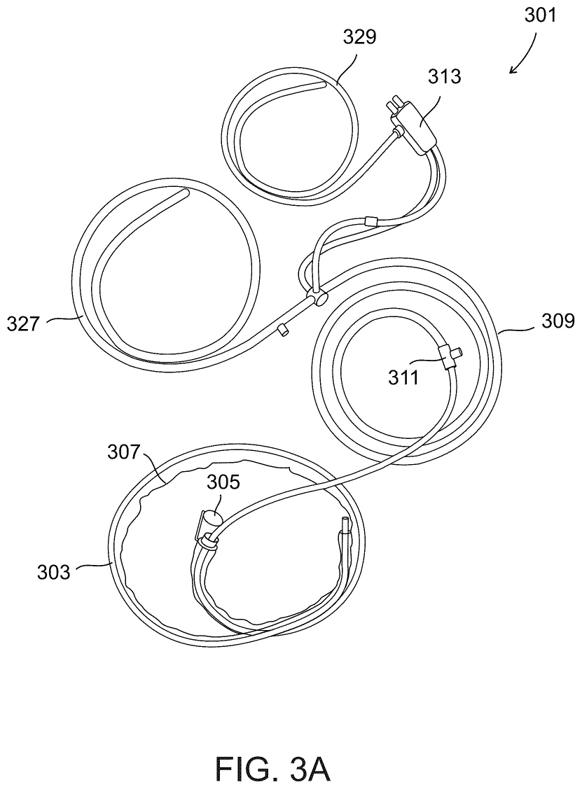

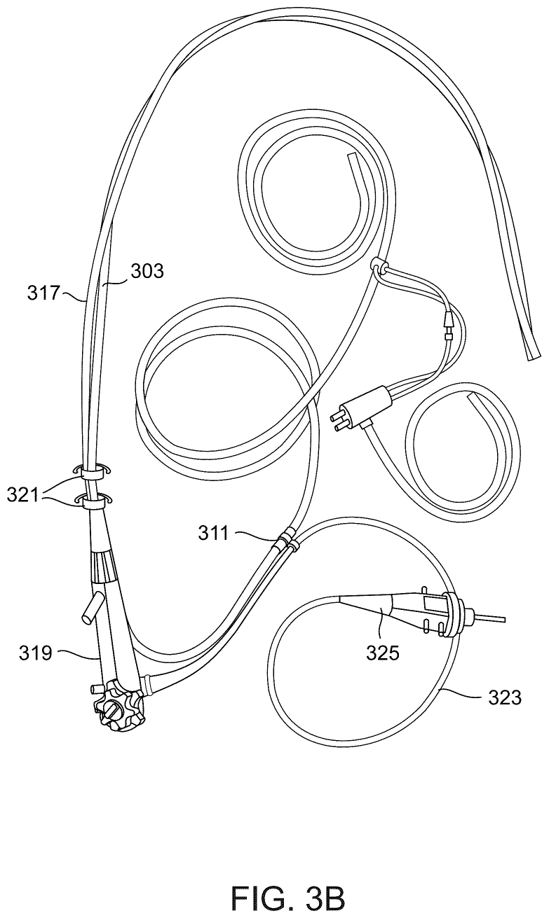

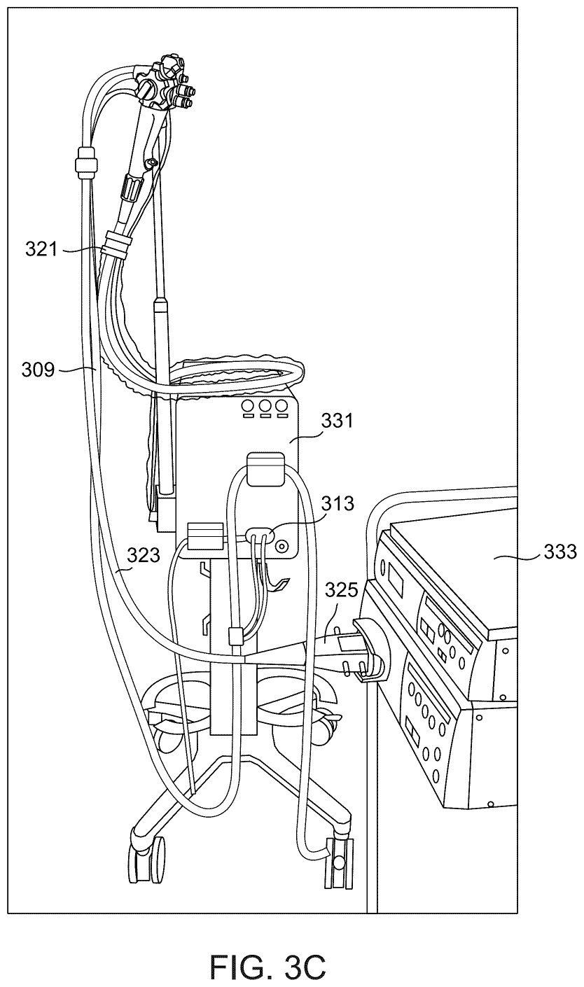

FIGS. 3A-C show the tubing system (FIG. 3A), the tubing system mounted onto a colonoscope (FIG. 3B), the tubing system and colonoscope connected to a tubing system working station and a colonoscope working station respectively (FIG. 3C), according to some embodiments.

In some embodiments, as shown for example in FIG. 3A, tubing system 301 comprises an insertion tube 303 configured to be inserted, at least in part, into the patient's body. In some embodiments, a coupling head 305 is mounted (optionally, integrally attached) at a distal end of the insertion tube, for coupling the tubing system insertion tube to the endoscope insertion tube. In some embodiments, a sleeve 307 (for example, a sheath formed of a thin material) is attached to head 305 and sized to extend (for example when pulled on) along at least a portion of the length of the insertion tube, for covering and holding together the tubing system insertion tube and the endoscope insertion tube.

In some embodiments, tubing system 301 comprises an umbilical tube 309 which is coupled by a connector 311 to the insertion tube 303. In some embodiments, umbilical tube 309 extends from the connector (proximally to the insertion tube) to an adaptor 313 for connecting the tubing system umbilical tube to the tubing system working station.

Exemplary dimensions of the tubing system components include: a length of the insertion tube 303 ranging between 1900 mm to 2500 mm, such as 2000 mm, 2150 mm, 2400 mm or intermediate, longer or shorter; a length of the umbilical tube 309 ranging between 1800-2200 mm, such as 1900 mm, 2000 mm, 2100 mm or intermediate, longer or shorter.

In some embodiments, a branch 327 extends from the tubing system for disposing of waste fluid and/or fecal matter sucked from the body lumen. In some embodiments, branch 327 passes through the working station (e.g. through an upper peristaltic pump of the working station) and opens into a waste bag or canister.

In some embodiments, a second branch 329 extends from adaptor 313 for mixing fluids (e.g. water) with air for irrigation purposes. In some embodiments, branch 329 passes through a pump of the working station (e.g. through a lower peristaltic pump of the working station) and is connected at its end to a source of the fluids (e.g. fluid bag, canister).

FIG. 3B shows the tubing system connected to a colonoscope. In some embodiments, as shown, tubing system insertion tube 303 extends adjacent and parallel to the colonoscope insertion tube 317. In some embodiments, a dual ring element 321 holds the two tubes together. In some embodiments, one of the rings is attached to sleeve 307 and is configured to pull the sleeve along the length of the insertion tubes, covering them by the sleeve; and the other ring serves as a clip which is attached to the tubing system insertion tube, and is configured to be clipped on the endoscope insertion tube.

In some embodiments, a proximal section of insertion tube 303 extends along and optionally past a control handle 319 of the colonoscope, ending at connector 311. In some embodiments, connector 311 couples the tubing system to the colonoscope umbilical tube 323 (the colonoscope umbilical tube extending from control handle 319 to an adaptor 325 for connecting the colonoscope to the colonoscope working station).

FIG. 3C shows the tubing system and colonoscope connected to a tubing system working station 331 and a colonoscope working station 333 respectively.

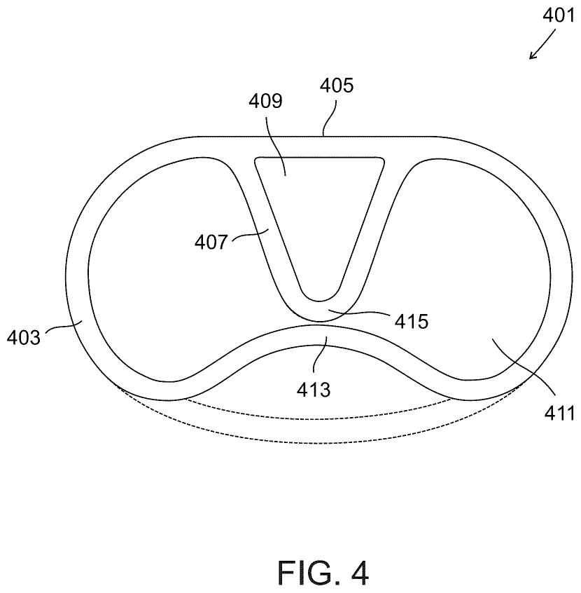

FIG. 4 is a cross section view of a multi-lumen insertion tube of a tubing system, according to some embodiments.

In some embodiments, insertion tube 401 comprises an outer wall 403 which normally assumes an oval or a partially oval shape. In some embodiments, the outer wall assumes an oval profile along at least 60%, 70%, 80% or intermediate, larger or smaller percentage of its length, and a straight (linear) profile (see 405) at the remaining portion of its length.

In some embodiments, insertion tube 401 includes an inner septum 407 shaped and positioned to divide the volume inside outer wall 403 into at least 2, at least 3, at least 4 or a higher number of separate lumens. In the example shown, inner septum divides the volume into a first lumen 409 and a second lumen 411. In some embodiments, first lumen 409 is used for irrigation and/or other introducing of fluids into the colon. In some embodiments, second lumen 411 is used for suction of material (such as of fecal matter and/or fluids from the colon), and/or for purging, for example for pushing away (e.g. distally) blockage inside lumen 411 or for example for pushing away an obstruction (partial or full) of a distal opening of insertion tube 401 such as by fecal material, tissue or other. In some embodiments, purging and suction are alternately applied through lumen 411, for example by reversing a direction of the pressure inside the tube, i.e. applying distally acting pressure during purging, and proximally acting pressure during suction.

In some embodiments, inner septum 407 is shaped and positioned to support at least a portion of outer wall 403 of the insertion tube from collapsing. In an example, when suction is applied through second lumen 411, a portion of outer wall 403 (see portion 413) may flex or bend inwards as a result of being pulled on by the suction. In such situation, septum 407 acts as support for the outer wall which prevents the outer wall from further collapsing inwardly. In some embodiments, septum 407 is sized and positioned so that when an outer wall portion bends and leans against the septum, at least 50%, 60%, 80%, 90% or intermediate, larger or smaller percentage of the second lumen's cross section remains open and non-collapsed, allowing the passage of material through.

In some embodiments, as shown, septum 407 comprises a triangular profile, with an inwards-directed apex 415 on which a bent portion of outer wall 403 may lean against. In some embodiments, apex 415 is substantially rounded.

In some embodiments, for example when purging is applied through second lumen 411, outer wall 403 expands radially outwardly, allowing for an increase in the cross section area of lumen 411.

In some embodiments, walls forming the insertion tube are relatively thin, for example having a thickness of 0.5 mm, 1 mm, 2 mm, 5 mm or intermediate, higher or lower thickness. Thin walls may contribute to the flexibility of the insertion tube, facilitating passing of the insertion tube through curvy narrow body lumens (such as the digestive tract).

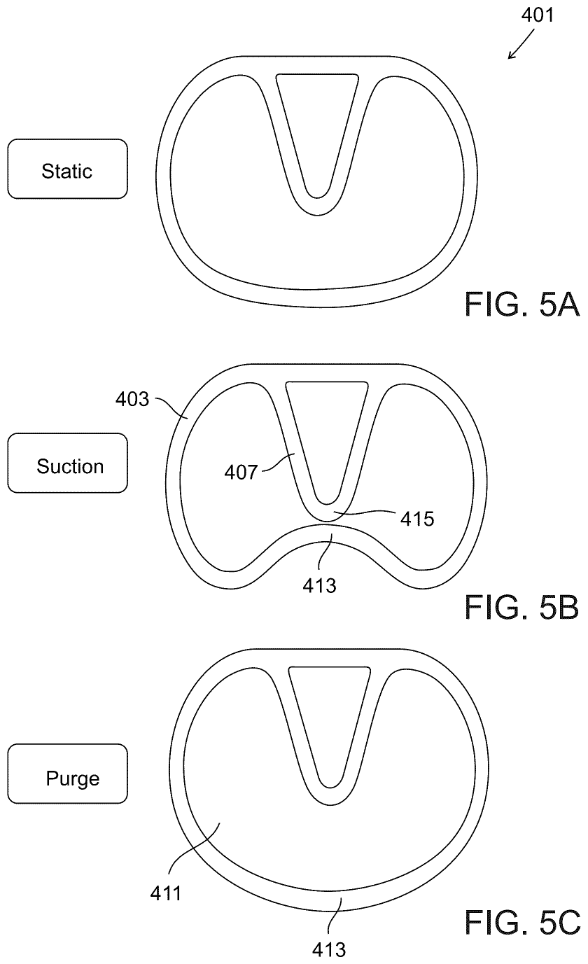

FIGS. 5A-C are cross section views of the multi-lumen insertion tube of FIG. 4 during static mode, suction mode, and purging mode, according to some embodiments. In FIG. 5A, insertion tube 401 is shown in a static mode, for example, at its initial configuration, e.g. when no suction or purging are applied therethrough. In FIG. 5B, portion 413 of the outer wall 403 is shown to partially collapse against apex 415 of the septum 407, which may occur for example in response to suction being applied through second lumen 411. In FIG. 5C, at least portion 413 is shown to expand radially outwardly, thereby increasing the effective cross sectional area of second lumen 411. This may occur, for example, in response to purging being applied through second lumen 411. In another example, in case of a blockage within second lumen 411, a portion (e.g. portion 413) of the outer wall can be pushed radially outwardly, potentially allowing for surrounding material to bypass the blockage and continue flowing through the second lumen 411.

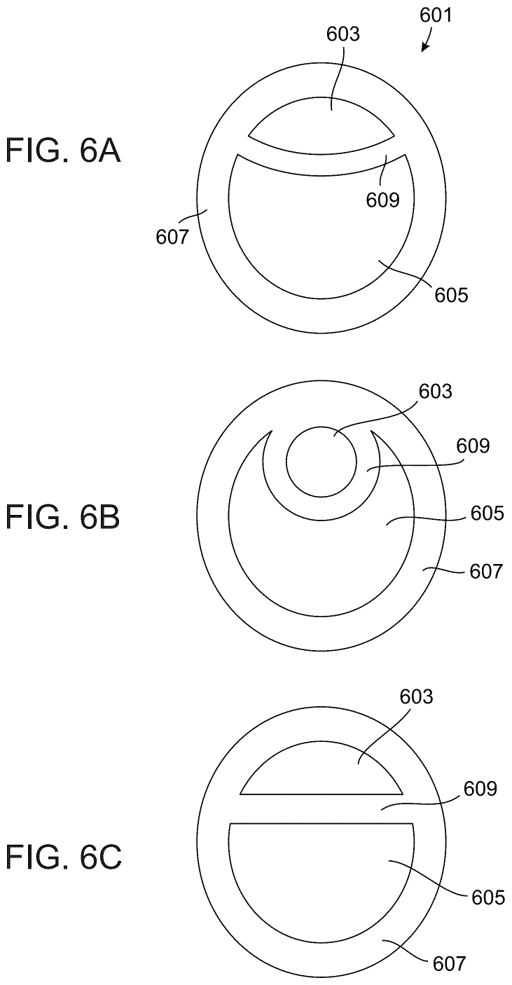

FIGS. 6A-C show various cross sections of a multi-lumen umbilical tube, according to some embodiments.

In some embodiments, as shown for example in FIG. 6A, umbilical tube 601 of the tubing system is internally divided into a plurality of inner lumens corresponding to the inner lumens of the insertion tube (e.g. as shown in FIG. 4). In some embodiments, a first lumen 603 is configured to be coupled in to first lumen 409 of the insertion tube, forming fluid communication with it; and a second lumen 605 is configured to be coupled to second lumen 411 of the insertion tube, forming fluid communication with it.

In some embodiments, coupling of the plurality of lumens of the insertion tube to the plurality of lumens of the umbilical tube is via a connector (not shown in this figure).

In some embodiments, as the umbilical tube extends only outside the patient's body, the outer wall 607 and/or the inner wall 609 forming a septum of the umbilical tube are thicker and more rigid than the walls forming insertion tube, potentially reducing a risk of collapse of the umbilical tube. In an example, walls of the umbilical tube have a thickness between 1.2-2 mm, 0.5-3 mm. 1-4 mm or intermediate, higher or lower thickness.

FIG. 6B shows an example of a multi-lumen umbilical tube in which first lumen 603 comprises a rounded, circular profile. Potential advantages of a rounded inner lumen may include simpler manufacturing and potentially a more rigid construction (defined by the rounded inner wall 609).

FIG. 6C shows an example of a multi-lumen umbilical tube in which first lumen 603 is semi-circular and is defined by inner wall 609 which extends as a direct linear septum between the opposite sides of outer wall 607.

In some embodiments, the umbilical tube cross sectional profile is different than a cross section of the insertion tube due to the different requirements of the umbilical tube, for example, the umbilical tube does not need to be as flexible as the insertion tube, does not need to provide for a similar extent of radial expansion as the insertion tube, and the like. This may enable using an umbilical tube of thicker walls and a simpler cross sectional profile.

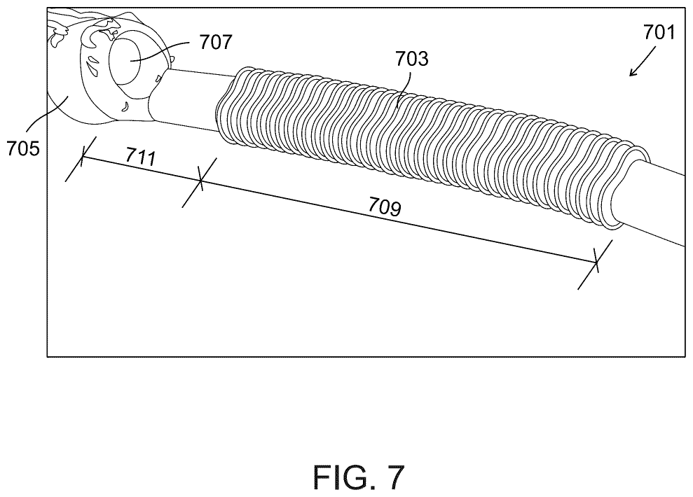

FIG. 7 shows a corrugated section of the multi-lumen insertion tube, according to some embodiments.

In some embodiments, an insertion tube 701 of the tubing system comprises an externally corrugated section 703, situated for example along a distal portion of the tube, in proximity to a distal head 705 of the insertion tube. (In some embodiments, as shown, head 705 defines a recess 707 in which a distal end of an endoscope insertion tube is received).

In an example, an axial length 709 of the corrugated section is between 50-200 mm, such as 75 mm, 100 mm, 125 mm or intermediate, longer or shorter. In some embodiments, the corrugated section starts at a distance 711 of, for example, between 20-80 mm from distal head 705, such as 25 mm, 40 mm, 60 mm or intermediate, longer or shorter.

In some embodiments, the ridges and grooves structure of the corrugated section provide axial extension and compression to an extent that may facilitate steering and bending of the insertion tube. In an example, the corrugated section is configured for full retroflexion (a U-shaped curve). Another potential advantage of the ridges and grooves structure of the corrugated section may include permitting local radial expansion, for example during purging, which may assist in clearing up clogged areas inside the insertion tube.

In some embodiments, the corrugated section is manufactured in a multi-stage process, involving controlled pressure and temperature conditions, which ensures the inner lumens of the insertion tube remain in their designated shape and unobstructed.

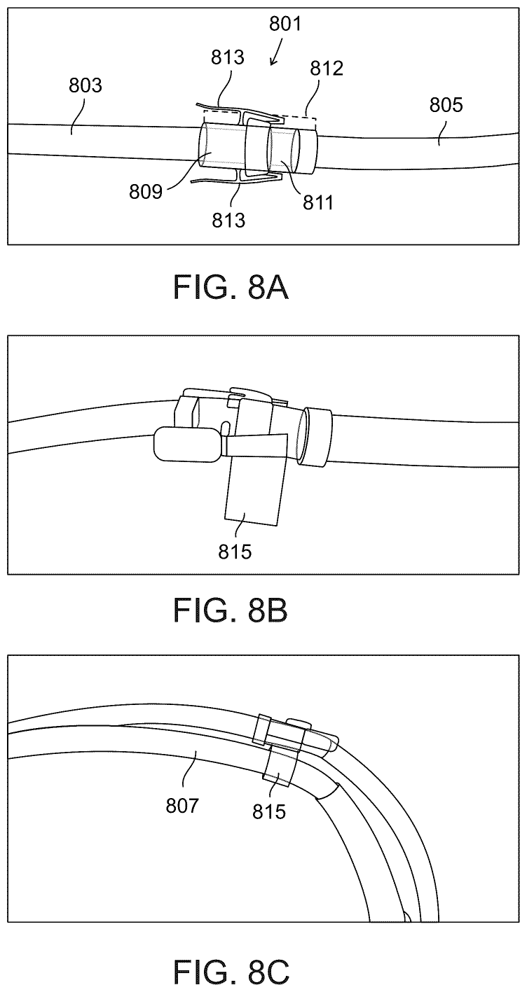

FIGS. 8A-C schematically show a connector for coupling a multi-lumen insertion tube to a multi-lumen umbilical tube and for mounting the assembled tubing system onto an endoscope, according to some embodiments.

FIGS. 8A-B are a front view and a side view of connector 801 which connects between a multi-lumen insertion tube 803, and a multi-lumen umbilical tube of the 805 of the tubing system, according to some embodiments. In some embodiments, as shown in FIG. 8C, connector 801 attaches the connected insertion and umbilical tubes to an endoscope 807.

In some embodiments, the connector is shaped to form fluid communication between the insertion tube and the umbilical tube. In some embodiments, the connector includes an interface which couples different cross section profiles of the insertion tube and the umbilical tube, for example coupling a first inner profile of the insertion tube (e.g. as shown in FIGS. 5A-C) with a second different inner profile of the umbilical tube (e.g. as shown in FIGS. 6A-C).

In some embodiments, connector 801 is transferable between an open configuration in which the tubes are inserted into (and/or otherwise attached to) designated portions 809, 811 of the connector body 812, optionally configured on opposing sides of the connector, and a closed configuration in which the tubes are sealingly held by the connector to each other, and optionally attached to the endoscope.

In some embodiments, connector 801 comprises wings 813 which are spread apart in the open configuration of the connector and closed towards body 812 of the connector in the closed configuration.

In some embodiments, connector 801 comprises a loop shaped attachment (e.g. a strap) 815 extending substantially perpendicularly to the connector body 812 for receipt of the endoscope 807 therethrough.

It is noted that some embodiments may not include a connector. For example, the insertion tube and the umbilical tube may be integrally and continuously formed as a single unit. Optionally, a cross section profile of such integral tube varies at one or more axial positions along the length (e.g. at the end of the segment which enters the patient body and the start of the segment which remains outside the patient body).

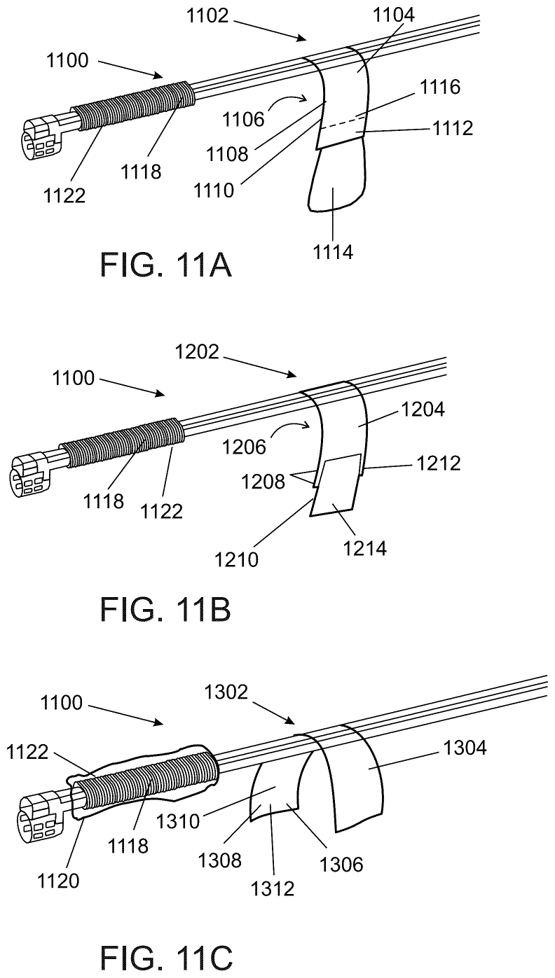

Alternatively, a multi-lumen insertion tube and/or a multi-lumen umbilical tube may be coupled to an endoscope by strips such as, for example, discussed herein with reference to FIGS. 11A-C. The strips may be similar to single-sided tape but may have a backing to cover the adhesive in the package. The backing may be peeled off (like in an adhesive bandage) for attaching the strip to the multi-lumen insertion/umbilical tube. One or more strips may be pre-attached in any of various locations, during a manufacturing process or the strips may be applied by a user, who decides on their location(s) along the endoscope and/or the multi-lumen insertion/umbilical tube. As the strip may be fairly thin and/or flimsy, an edge of the strip may include a rigid piece of plastic at an end thereof to aid in wrapping the strip around the endoscope and/or multi-lumen insertion/umbilical tube.

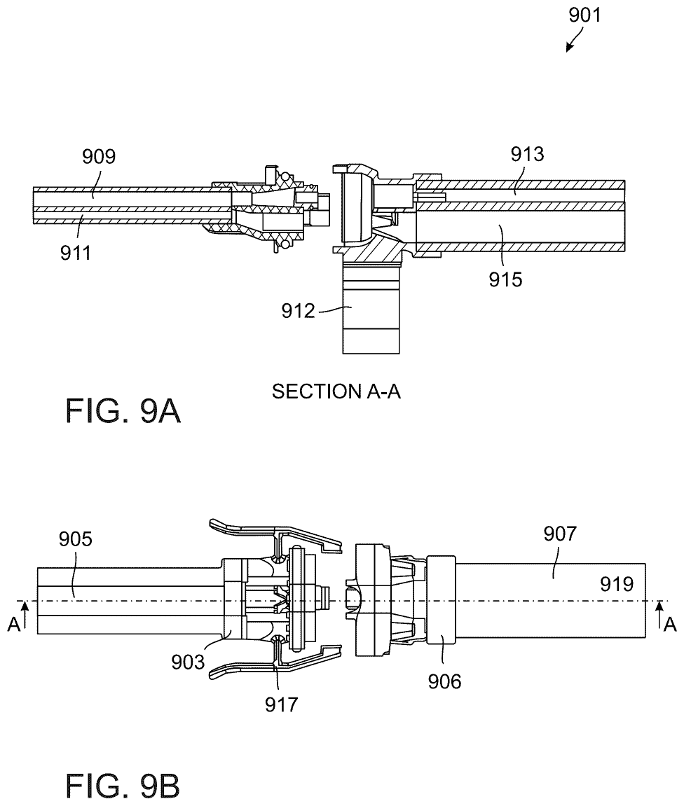

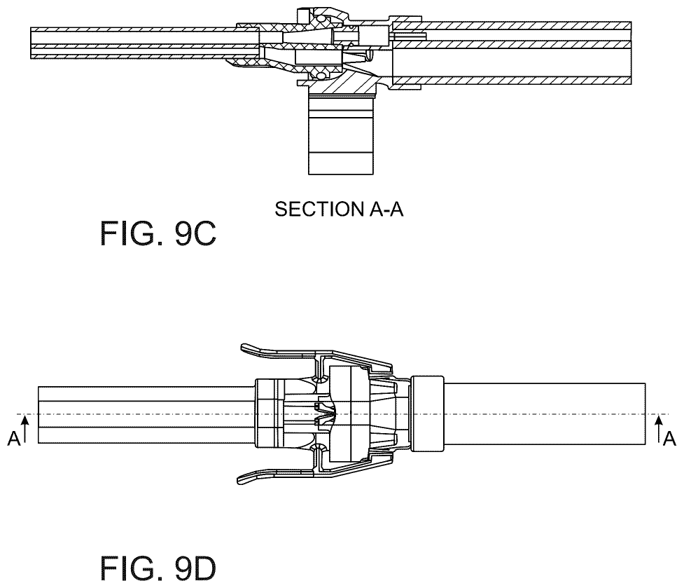

Once the strip is wrapped around the endoscope and/or multi-lumen insertion/umbilical tube, and the endoscope has been secured to the multi-lumen insertion/umbilical tube, the edge of the strip having the rigid piece of plastic may be cut off. Alternatively, the strip may include perforations to facilitate tearing off of the rigid plastic. An alternative configuration may include strips having a rectangular or an oval cross-sectional profile, the strips provided on a backing sheet, where a user may peel the strip off the backing sheet and wrap the strips around the multi-lumen insertion/umbilical tube and the endoscope at various points. FIGS. 9A-D are detailed views of a connector 901 for coupling a multi-lumen insertion tube to a multi-lumen umbilical tube and for mounting the assembled tubing system onto an endoscope, shown in an open state (FIGS. 9A-B) and a closed state (FIGS. 9C-D).

In some embodiments, connector 901 includes a first portion 903 in which the insertion tube 905 is received, and a second portion 906 in which the umbilical tube 907 is received. In some embodiments, one of the portions (for example, the first portion 903) is a male connector and the second portion (906) is a female connector.

In some embodiments, as shown in the longitudinal cross section views of FIGS. 9A, 9C, inner lumens 909, 911 of the insertion tube are interfaced with inner lumens 913, 915 of the umbilical tube respectively. In some embodiments, lumen 909 which optionally functions for irrigation purposes is brought into fluid communication with lumen 913 which also serves for irrigation; and lumen 911 which optionally function for suction/evacuation purposes is brought into fluid communication with lumen 915 which also serves for suction/evacuation.

In some embodiments, as shown for example in FIGS. 9B, 9D, the connector comprises side wings 917 which extend radially outwardly from a long axis 919 of the tubes/connector in an open state of the connector, and are pushed radially inwardly in the closed state of the connector, for sealing and maintaining hold of the connection between the insertion tube and umbilical tube.

In some embodiments, an endoscope is introduced into a space defined by the loop shaped attachment 912 which extends sideways relative to the tubes/connector long axis.

In some embodiments, the connector inner geometry is formed with smooth walls and without protrusions or other surface irregularities so as to potentially reduce or avoid blockages and allow uninterrupted flow at the interface between the insertion tube and umbilical tube.

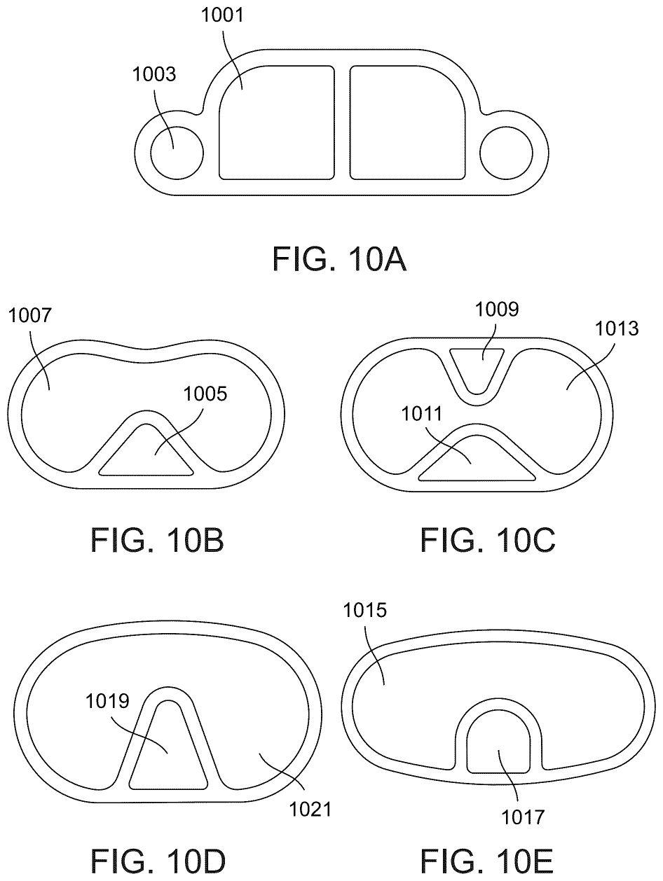

FIGS. 10A-E are alternative cross section profiles of a multi lumen insertion tube, according to some embodiments.

The example of FIG. 10A includes a middle partition which divides the tube cross section into two substantially square shaped lumens 1001 and two rounded (e.g. circular) lumens 1003, each located laterally to the square shaped lumen. In some embodiments, the square shaped lumens 1001 serve for suction/evacuation, and the rounded lumens 1003 serve for irrigation. A potential advantage of square shaped lumens may include a more rigid construction which may be at reduced risk of collapse, but on the other hand, may affect the tube's bending ability.

The example of FIG. 10B includes a substantially triangular lumen 1005 and a remaining lumen 1007 defined by a substantially elliptic outer wall. Optionally lumen 1005 serves for irrigation while the remaining lumen 1007 serves for suction/irrigation.

The example of FIG. 10C includes two triangular lumens 1009, 1011, optionally of various sizes, which are optionally located opposite each other within an outer lumen 1013 defined by a substantially elliptic outer wall.

The example of FIG. 10D is generally similar to the one shown in FIGS. 5A-C, but with different proportions between the inner triangular lumen 1019 and the outer lumen 1021 defined by a substantially elliptic outer wall. In this example, outer lumen 1021 includes a larger cross sectional area which may provide for a higher rate and/or volume of suction of matter therethrough.

The example FIG. 10E includes a lumen 1015 defined by a substantially elliptic outer wall and an inner lumen 1017 bordered by a partially arched wall.

Referring now to FIGS. 11A-C there are shown an endoscope 1100 and various strips for connecting the endoscope to a multi-lumen insertion tube or a multi-lumen umbilical tube, according to some embodiments. It should be understood that, if desired, the strips may be alternatively provided on a multi-lumen insertion tube or a multi-lumen umbilical tube, for connecting the multi-lumen insertion tube or multi-lumen umbilical tube to an endoscope.

While only one strip of each type is shown in each of FIGS. 11A-C, it will be appreciated that, if desired, any number of strips may be provided on the endoscope 1100, at various locations along the endoscope, to attach a multi-lumen insertion/umbilical tube to the endoscope. Similarly, at least one strip may be provided on a colonoscope, at various locations therealong, to attach a multi-lumen insertion/umbilical tube to the colonoscope, according to some embodiments. For example, multiple strips may be utilized and, depending on the type and length of the endoscope/colonoscope, there may be, for example, from 3 to 10 strips evenly spaced apart along the endoscope/colonoscope. According to some embodiments, the strips may be positioned along the endoscope/colonoscope at any suitable distance apart such as, for example, distances in the range of 10-30 cm apart, or at any other suitable distance apart, according to some embodiments. Alternatively, the strips may be non-uniformly spaced along the endoscope/colonoscope, according to some embodiments, for example, with more strips at a distal area of the endoscope/colonoscope which generally bends more than a proximal portion of the endoscope/colonoscope, which is generally straighter. For example, the strips may be positioned along the endoscope/colonoscope at varying distances apart ranging from, for example, 5-40 cm apart, or at any other suitable range of distances apart

While the ensuing description may describe attaching a multi-lumen insertion/umbilical tube to an endoscope, it is to be understood that the multi-lumen insertion/umbilical tube may similarly be attached to a colonoscope.

The use of at least one strip for attaching a multi-lumen insertion/umbilical tube to an endoscope may also aid in preventing kinking of the tubing and may case insertion of an insertion tube/endoscope while preventing the insertion tube from bending away from the endoscope.

As seen in FIG. 11A, endoscope 1100 is provided with a strip 1102 having an elongate shape, according to some embodiments. The strip may be formed of any suitable material. Suitable materials may be selected from polymers such as, for example polyurethane, PVC, a polyester, polyethylene and polypropylene.

The strip may be of any length and width suitable for the type of endoscope and insertion tube being attached together. For example, according to some embodiments, a strip may be from 3-5 cm long and from 2-5 cm wide.

According to some embodiments, the strip 1102 may be premounted on the endoscope 1100 or, alternatively, according to some embodiments, may be attached to the endoscope by a user. The strip 1102 is preferably disposable, according to some embodiments.

The strip 1102 may include a first side 1104 and a second side 1106. According to some embodiments, the strip 1102 may be provided with an adhesive layer 1108 on second side 1106, and a backing 1110 may be positioned over the adhesive layer 1108, the backing to be removed when the strip 1102 is to be attached to the endoscope. The adhesive layer may include any suitable adhesive such as, for example, acrylic, or may include any silicone-based or other medical grade adhesive. The backing may be formed of any suitable material such as, for example, paper optionally having a polymer coating which could also be a fluoropolymer, polyurethane, PVC, a polyester, polyethylene, and polypropylene.

An end portion 1112 of strip 1102 may be provided with a tab 1114. Tab 1114 may be rigid and may aid in wrapping the strip 1102 around the endoscope 1100. The strip may be provided with a perforation 1116 near end portion 1112, to aid a user in holding the strip 1102 and wrapping the strip around the endoscope. The tab 1114 may be removed by tearing strip 1102 along perforation 1116, as discussed herein. If desired, strip 1102 may be wrapped around and/or adhered to a multi-lumen insertion/umbilical tube (not shown) in order to attach the endoscope 1100 thereto.

According to some embodiments, endoscope 1100 may include at least one bendable portion, for example accordion portion 1118, to facilitate navigation of the endoscope 1100 through winding passageways of a lumen such as, for example, a colon or small intestine. An accordion portion 1118 may be located at a distal end of the endoscope 1100, as shown in each of FIGS. 11A-C. Alternatively or additionally, at least one bendable portion may be located at any suitable portion along the endoscope 1100 such as, for example, between strips

It should be understood that, alternatively, any other suitable type of bendable portion may be provided instead of accordion portion 1118, according to some embodiments.

FIG. 11B shows endoscope 1100 having a strip 1202 according to some embodiments. Strip 1202 has a first side 1204 and a second side 1206, the strip provided with a tab 1214 attached to an end portion 1212 of strip 1202. While strip 1202 is shown having tab 1214 attached to first side 1204 of the strip, alternatively, if desired, a tab may be attached to second side 1206 of the strip. According to some embodiments, tab 1214 may be provided with an adhesive layer 1208. A backing 1210 may be positioned on adhesive layer 1208. According to some embodiments, backing 1210 may be removed before the strip 1202 is to be attached to the endoscope 1100. Optionally, adhesive layer 1208 may continue from tab 1214 onto at least a portion of second side 1206 of strip 1202. The strip 1202 may be wrapped around the endoscope 1100 and may be secured to, for example, a multi-lumen insertion/umbilical tube, with the adhesive layer 1208 on tab 1214 and, optionally, also on second side 1206 of strip 1202. If desired, according to some embodiments, strip 1202 may be wrapped around and/or adhered to a multi-lumen insertion/umbilical tube (not shown) in order to attach the endoscope thereto.

FIG. 11C shows endoscope 1100 having a strip 1302 according to some embodiments. Strip 1302 has a first side 1304 and has a second side 1306 provided with an adhesive layer 1308 at least on an end portion 1312 of strip 1302. A backing 1310 is positioned on the adhesive layer 1308, the backing to be removed when the strip 1302 is to be attached to the endoscope 1100. If desired, strip 1302 may be wrapped around and/or adhered to a multi-lumen insertion/umbilical tube (not shown) in order to attach the endoscope thereto.

The endoscope 1100 may be provided with a generally tubular cover or sleeve 1120, over accordion portion 1118, according to some embodiments. Sleeve 1120 may be formed of any suitable material such as, for example, a polymer. Ends of sleeve 1120 may be attached to endoscope 1100 by any suitable method such as, for example, welding or adhesive, the sleeve extending over accordion portion 1118. The sleeve may be wide enough such that portions of the endoscope 1100 adjacent the accordion portion 1118 fit inside the sleeve. Sleeve 1120 may have a similar configuration and structure to that of sleeve 307 (FIG. 3A), but sleeve 1120 is generally shorter, yet long enough to cover accordion portion 1118.

By providing endoscope 1100 with sleeve 1120 positioned over accordion portion 1118, the endoscope is provided with a smoother outer surface at the locations of the accordion portion, in particular, at the locations of ridges 1122 of the accordion portion. This may facilitate insertion of the endoscope 1100 into passageways of a lumen such as, for example, a colon or small intestine, and thereby prevent or reduce trauma that may be caused by ridges 1122 or other portions of accordion portion 1118 rubbing against tissue of the lumen.

While sleeve 1120 is shown in the embodiment of FIG. 11C, it will be appreciated by persons skilled in the art that, if desired, any accordion portion of any of the herein-discussed embodiments may be covered by a sleeve.

The terms “comprises”, “comprising”, “includes”, “including”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

As used herein, the term “treating” includes abrogating, substantially inhibiting, slowing or reversing the progression of a condition, substantially ameliorating clinical or aesthetical symptoms of a condition or substantially preventing the appearance of clinical or aesthetical symptoms of a condition.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

It is the intent of the applicant(s) that all publications, patents and patent applications referred to in this specification are to be incorporated in their entirety by reference into the specification, as if each individual publication, patent or patent application was specifically and individually noted when referenced that it is to be incorporated herein by reference. In addition, citation or identification of any reference in this application shall not be construed as an admission that such reference is available as prior art to the present invention. To the extent that section headings are used, they should not be construed as necessarily limiting. In addition, any priority document(s) of this application is/are hereby incorporated herein by reference in its/their entirety.

1. A multi-lumen insertion tube for use with an endoscope, comprising:
a circumferential outer wall defining a main inner lumen;
a plurality of inner septa dividing said main inner lumen into a plurality of separate inner lumens;
wherein said circumferential outer wall is configured to expand radially outwardly when positive pressure is applied through the insertion tube, and to at least partially collapse against at least one of said inner septa when negative pressure is applied through the insertion tube, such that further collapse of the circumferential wall is prevented by said at least one of said inner septa.

2. The multi-lumen insertion tube according to claim 1, wherein said at least one of said inner septa defines a triangular wall, and said circumferential outer wall leans against an apex of said triangular wall when negative pressure is applied through the insertion tube.

3. The multi-lumen insertion tube according to claim 2, wherein said triangular wall is formed of wall segments short enough to be substantially undeformable under said negative pressure.

4. The multi-lumen insertion tube according to claim 1, wherein said circumferential outer wall is configured to expand radially outwardly when positive pressure is applied through said insertion tube.

5. The multi-lumen insertion tube according to claim 1, wherein said plurality of inner lumens include at least a first inner lumen and a second inner lumen, wherein flow in each of said first and second inner lumens is independently controlled.

6. The multi-lumen insertion tube according to claim 5, wherein said first inner lumen serves for irrigation and said second inner lumen serves for suction.

7. The multi-lumen insertion tube according to claim 1, wherein a cross section area of said multi-lumen insertion tube is sized for insertion into a colon.

8. The multi-lumen insertion tube according to claim 1, wherein a thickness of said circumferential outer wall is between 0.5 mm and 1 mm and a thickness of said inner septa is between 0.3 mm and 0.8 mm.

9. The multi-lumen insertion tube according to claim 1, wherein said circumferential outer wall comprises a corrugation along at least a segment of the length of the multi-lumen insertion tube.

10. A tubing system for use with an endoscope, comprising:
a multi-lumen insertion tube according to claim 1, the multi-lumen insertion tube shaped and sized for insertion into a body lumen;
a multi-lumen umbilical tube configured for extending externally to the body, the multi-lumen umbilical tube comprising a plurality of inner lumens; and
a coupling interface which couples a proximal end of said insertion tube to a distal end of said umbilical tube, the coupling interface shaped to fluidly connect said plurality of inner lumens of said insertion tube to said plurality of inner lumens of said umbilical tube.

11. The tubing system according to claim 10, wherein a proximal end of said multi-lumen umbilical tube is configured to connect to a pressure source which supplies positive and/or negative pressure.

12. The tubing system according to claim 10, wherein said multi-lumen umbilical tube is larger in cross-sectional area than said multi-lumen insertion tube.

13. The tubing system according to claim 10, wherein said coupling interface is formed as a connector comprising a first portion in which said proximal end of said insertion tube is received, and a second portion in which said distal end of said umbilical tube is received.

14. The tubing system according to claim 10, further comprising a loop shaped attachment extending laterally outwardly from said coupling interface for insertion of an endoscope therethrough.

15. The tubing system according to claim 10, including at least one adhesive strip, each said adhesive strip configured for attaching said tubing system to an endoscope at a location along said tubing system, wherein at said location at least one of:
said adhesive strip is wrapped at least partly around a first one of said tubing system and said endoscope; and
said adhesive strip is adhered to a second one of said tubing system and said endoscope.

16. The tubing system according to claim 10, wherein at least one of said multi-lumen insertion tube and said endoscope includes a bendable portion, said bendable portion provided with an outer sleeve configured to provide said at least one of said multi-lumen insertion tube and said endoscope with a smooth outer surface at a location of said bendable portion.

17. A method of setting up a tubing system for use with an endoscope, the tubing system comprising a multi-lumen insertion tube having a plurality of separate inner lumens, the multi-lumen insertion tube configured to connect at its proximal end to a multi-lumen umbilical tube, the method comprising:
aligning and coupling said multi-lumen insertion tube to an insertion tube of said endoscope;
connecting, via a connector, said multi-lumen insertion tube to said multi-lumen umbilical tube;
attaching the connector to said endoscope; and
connecting said multi-lumen umbilical tube to a working station of said endoscope.

18. The method according to claim 17, wherein said aligning and coupling said multi-lumen insertion tube to said insertion tube of said endoscope comprises one or both of holding, via a holder, the distal ends of said multi-lumen insertion tube and said endoscope insertion tube together; and covering said multi-lumen insertion tube and said endoscope insertion tube along their length by a sleeve which holds them together.

19. The method according to claim 17, wherein said attaching said connector to an endoscope comprises placing a loop shaped attachment extending from said connector around said endoscope.

20. The method according to claim 17, including attaching said tubing system to said endoscope at at least one location along said tubing system, wherein at each said location said attaching said tubing system to said endoscope includes at least one of:
wrapping an adhesive strip at least partly around a first one of said tubing system and said endoscope; and
adhering said adhesive strip to a second one of said tubing system and said endoscope.

21. (canceled)

22. A method of operating a tubing system coupled to an endoscope, the tubing system including a multi-lumen insertion tube having a plurality of separate inner lumens, the method comprising:
introducing an insertion tube of said endoscope along with said multi-lumen insertion tube of said tubing system into the GI tract, side-by-side;
applying one of suction and purging via said multi-lumen insertion tube; wherein during suction, inner septa of the multi-lumen insertion tube prevent said multi-lumen insertion tube from collapsing, and wherein during purging an outer wall of said multi-lumen insertion tube expands radially outwardly.

23. The method according to claim 22, further comprising removing said endoscope insertion tube and said multi-lumen insertion tube from the GI tract, detaching said tubing system from said endoscope insertion tube and disposing of said tubing system.

24-26. (canceled)

27. The multi-lumen insertion tube according to claim 9, wherein said corrugation includes ridges and grooves shaped and sized to provide for local expansion at multiple axial locations along said segment.

28. The multi-lumen insertion tube according to claim 27, wherein said segment is more flexible than one or more other longitudinal segments of said insertion tube.

29. The multi-lumen insertion tube according to claim 9, wherein when said multi-lumen insertion tube is inserted alongside an insertion tube of said endoscope, said segment is axially aligned with a bendable section of said endoscope insertion tube.

30. The multi-lumen insertion tube according to claim 27, wherein said multi-lumen insertion tube is configured to be inserted alongside an insertion tube of an endoscope, said multi-lumen insertion tube attached to said endoscope insertion tube at at least one location along said multi-lumen insertion tube, wherein at each said location at least one of:
an adhesive strip is wrapped at least partly around a first one of said multi-lumen insertion tube and said endoscope insertion tube; and
said adhesive strip is adhered to a second one of said multi-lumen insertion tube and said endoscope insertion tube.

31. The multi-lumen insertion tube according to claim 30, wherein at least one of said multi-lumen insertion tube and said endoscope insertion tube includes a bendable portion, said bendable portion provided with an outer sleeve configured to provide said at least one of said multi-lumen insertion tube and said endoscope insertion tube with a smooth outer surface at a location of said bendable portion.

32. The multi-lumen insertion tube according to claim 1, wherein said plurality of inner septa extend along the entire length of said insertion tube, said plurality of inner septa dividing said main inner lumen along said entire length of said insertion tube into the plurality of separate inner lumen.

33. The multi-lumen insertion tube according to claim 1, wherein said plurality of separate inner lumen include at least first and second inner lumens; and
wherein first and second pressures are supplied through said first and second inner lumens, respectively, and wherein said first and second pressures differ in at least one of magnitude and direction.

34. The multi-lumen insertion tube according to claim 1, wherein said multi-lumen insertion tube is configured to be inserted alongside an insertion tube of an endoscope, said multi-lumen insertion tube attached to said endoscope insertion tube at at least one location along said multi-lumen insertion tube, wherein at each said location at least one of:
an adhesive strip is wrapped at least partly around a first one of said multi-lumen insertion tube and said endoscope insertion tube; and
said adhesive strip is adhered to a second one of said multi-lumen insertion tube and said endoscope insertion tube.
